(19) United States
(12) Patent Application Publication
(10) Pub. No.: US 2009/0171115 A1
(43) Pub. Date: Jul. 2, 2009

(54) PREPARATION OF ION DISSOCIATION FUNCTIONAL MOLECULE AND PREPARATION OF RAW MATERIAL MOLECULE THEREOF

(75) Inventor: Yongming Li, Kanagawa (JP)

Correspondence Address: K&L Gates LLP, P. O. BOX 1135, CHICAGO, IL, 60690, US

(73) Assignee: SONY CORPORATION, Tokyo (JP)

(21) Appl. No.: 11/911,680

(22) PCT Filed: Apr. 5, 2006
(86) PCT No.: PCT/JP2006/307192

§ 371 Date: Dec. 12, 2008

(30) Foreign Application Priority Data
May 11, 2005 (JP) 2005-138057

(51) Int. Cl. C07C 309/24

(52) U.S. Cl. 562/42; 562/41

(57) ABSTRACT

Disclosed is a method of preparing an ion dissociation functional molecule which is chemically and thermally stable under the operating conditions required in an electrochemical system such as a fuel cell and which is suitable for use as a material of, for example, a proton conductor used in a fuel cell, in a higher yield and more easily, more efficiently, more inexpensively and more safely, than by the existing art. In a second step of reacting AgOOCCF₂SO₂F with iodine to synthesize a raw material molecule ICF₂SO₂F, the reactants mixed in equimolar relation are reacted with each other at 110° C., which is higher than that in the existing art, to thereby enhance the production efficiency of the raw material molecule, then a mixed gas of the thus produced raw material molecule with carbon dioxide is pre-cooled in an exhaust passage kept at −15° C., followed by trapping the raw material molecule by a trapping vessel cooled with dry ice, whereby the trapping efficiency for the raw material molecule is enhanced. In a third step which would be carried out using an autoclave in the existing art, a fullerene and the raw material molecule are reacted by use of a reaction solvent having a boiling point comparable to or higher than the reaction temperature, whereby the reaction is permitted to take place at normal pressure or in a slightly pressurized condition.

### TECHNICAL FIELD

The present invention relates to a method of preparing an ion dissociation functional molecule suitable as a material of, for example, a proton conductor used in a fuel cell, and to a method of preparing a raw material molecule of the ion dissociation functional molecule.

### BACKGROUND ART

One of the proton conductors most widely used in solid polymer electrolyte type fuel cells and the like is Nafion (tradename of a perfluorosulfonic acid based resin, produced by DuPont), which is a perfluorinated sulfonic acid based polymer resin having the structure represented by the following chemical formula IV:

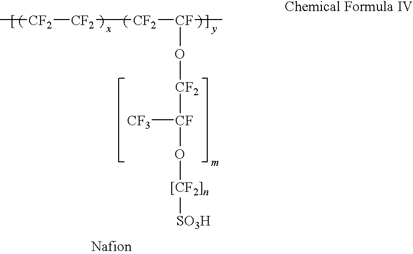

The molecular structure of Nafion includes two sub-structures intrinsically different in properties, namely, (1) a single perfluorinated main chain which constitutes a hydrophobic molecular skeleton, and (2) a perfluorinated side chain which contains a hydrophilic sulfonic group and which functions as a proton donating site. This structure, containing no unsaturated bond and having been perfluorinated, promises thermal and chemical stability. In a dry atmosphere or at high temperatures, however, the water occluded in the resin and needed for development of proton conductivity is liable to be lost, resulting in a lowering in proton conductivity.

On the other hand, the present applicant has already shown, in WO 01/06519 (pp. 6 to 11, FIGS. 1 and 2) to be described later (hereinafter referred to as Patent Document 1), that materials composed mainly of a carbon cluster derivative obtained by introduction of a proton dissociating group such as the hydrogen sulfate ester group (—OSO₃H) or the sulfonic group (—SO₃H) into a carbon cluster such as a fullerene, as exemplified by (A) and (B) of FIG. 5, is capable of exhibiting proton conductivity in a solid structure. Besides, in Japanese Patent Laid-open No. 2003-303513 (pp. 7 to 10, FIGS. 1 and 4) (hereinafter referred to as Patent Document 2), the present applicant has exemplified the compounds represented by (C) and (D) of FIG. 5, as fullerene derivatives which have proton conductivity. The proton dissociating group may be bonded directly to the fullerene nucleus, as in the cases of (A) and (B) of FIG. 5, or may be linked indirectly to the fullerene nucleus through any of various spacer groups, as in the cases of (C) and (D) of FIG. 5. Where the amount of water contained in the solid structure is optimized, these compounds can exhibit a proton conductivity of more than 10⁻² S/cm.

Thus, the present applicant has found out that a material for conduction of ions such as protons can be obtained by introduction of a functional group to a carbon cluster such as a fullerene. When the proton-conductive function present in such a material is applied, for example, to an electrochemical system such as a fuel cell, the material is required to be stable both chemically and thermally under the conditions needed for the electrochemical system.

FIG. 6 shows chemical formulas representing the structures of proton-conductive fullerene derivatives which have excellent chemical and thermal stability, as reported by the present applicant in Patent Document 2 and Japanese Patent Laid-open No. 2005-68124 (pp. 10 and 11 to 13, FIG. 1) (hereinafter referred to as Patent Document 3), so as to be able to meet the above-mentioned requirement. In the proton dissociating functional molecules exemplified in FIG. 6, the proton dissociating group is linked to the fullerene nucleus through a spacer group, instead of being bonded directly to the fullerene nucleus, so that the influence of the unsaturated bonds constituting the fullerene nucleus would not be exerted on the proton dissociating group. In addition, the spacer group is a group which includes, for example, an alkylene group with its hydrogen atoms at least partly substituted with fluorine atom(s), and which has been chemically inactivated and been strengthened in heat resistance. Therefore, the proton dissociating functional molecules shown in FIG. 6 exhibit excellent chemical stability and heat resistance.

Particularly, the proton dissociating functional molecule shown in (E) of FIG. 6 corresponds to poly(difluorosulfomethyl)fullerene C₆₀, in which n sulfonic groups —SO₃H (n is a natural number) are each linked to the fullerene nucleus through the difluoromethylene group —CF₂—. The difluoromethylene group is chemically inactive and highly heat-resistant, and, therefore, this proton dissociating functional molecule is the most stable, both thermally and chemically, of the molecules shown in FIG. 6. Moreover, the difluoromethylene group has the minimum size required of the spacer group, making it possible to introduce many proton dissociating groups to one fullerene molecule; therefore, it is possible to enhance the density of the proton dissociating groups and, thereby, to realize a high proton conductivity even under comparatively low humidity conditions.

Incidentally, in order to accurately represent the poly(difluorosulfomethyl)fullerene C₆₀, it may be desirable to represent it as in Reference Drawing given in round-edged parentheses. However, the positions where the difluoromethylene groups are bonded to the fullerene nucleus are unclear, and the representation in Reference Drawing is too complicated. Therefore, the fullerene derivatives are herein represented in a simplified form as in (E) of FIG. 6, instead of adopting the representation in Reference Drawing. The simplified representation applies not only to (A) to (D) of FIG. 5 and (A) to (D) of FIG. 6 but also to all the fullerene derivatives mentioned herein.

In addition, the term “proton dissociating group” in the above description means a functional group from which a hydrogen atom can be ionized and liberated as proton (H⁺). This definition also applies in the present invention. Besides, a functional group from which a metal ion or the like can be liberated as an ion will hereinafter be referred to as “ion dissociating group”. Further, the “functional group” includes not only the meaning of an atomic group having only one bond but also the meaning of an atomic group having two or more bonds. The “functional group” may be bonded to an end of a molecule, or may be present in a molecular chain.

FIG. 7 shows a flowchart of the synthesis process of a proton dissociating functional molecule described in Patent Document 3. FIG. 7 shows an example in which the fullerene molecule is C₆₀ and the raw material molecule to be reacted with the fullerene molecule is difluoroiodomethanesulfonyl fluoride: ICF₂SO₂F.

In the synthesis flow shown in FIG. 7, first, the raw material molecule ICF₂SO₂F is synthesized in the first and second steps in the former steps [refer to Chen Qing-Yuu, ACTA. CHIMICA. SINICA., 48 (1990), 596 (hereinafter referred to as Non-patent Document 1) and Patent Document 3].

In the first step, silver difluoro(fluorosulfonyl)acetate: AgOOCCF₂SO₂F is synthesized from difluoro(fluorosulfonyl)acetic acid: HOOCCF₂SO₂F by the following reaction.

Ag₂CO₃+2HOOCCF₂SO₂F→H₂O+CO₂+2AgOOCCF₂SO₂F

Specifically, 5.0 g (18.2 mmol) of silver carbonate is dispersed in diethyl ether at room temperature, and, while stirring the dispersion, 6.5 g (36.3 mmol) of difluoro(fluorosulfonyl)acetic acid is slowly added dropwise thereto. After the dropwise addition, stirring is continued for about one day at room temperature, to effect reaction. After the reaction is over, the reaction mixture is filtered to remove unreacted silver carbonate therefrom, followed by evaporating off the ether, whereon a while solid matter is obtained. The solid matter is recrystaliized from a mixed solvent of diethyl ether and hexane, whereon while needle-like crystals of pure silver difluoro(fluorosulfonyl)acetate is obtained, in an amount of 9.6 g, the yield being 93%.

Next, in the second step, iodine is let act on silver difluoro(fluorosulfonyl)acetate: AgOOCCF₂SO₂F to synthesize difluoroiodomethanesulfonyl fluoride: ICF₂SO₂F by the following reaction.

AgOOCCF₂SO₂F+I₂→ICF₂SO₂F+CO₂+AgI

Specifically, a reaction equipment provided with a cooling pipe so that a reaction mixture in a reaction vessel can be subjected directly to distillation is assembled. The reaction vessel is charged with 7.2 g (26.2 mmol) of silver difluoro(fluorosulfonyl)acetate and 10 g (78.6 mmol) of iodine, followed by heating at 100° C., whereon the desired difluoroiodomethanesulfonyl fluoride is distilled through the cooling pipe of the distilling apparatus, and it is recovered by use of an iced bath. The amount of the product obtained is 3.3 g, the yield being 48%.

The major problem in the former steps is the low yield upon the second step.

Subsequently, in the third to fifth steps in the latter steps, the raw material molecule ICF₂SO₂F is let act on fullerene C₆₀, the resulting precursor molecule is then hydrolyzed, to obtain a proton dissociating functional molecule, thereby synthesizing poly(difluorosulfomethyl)fullerene C₆₀ shown in (E) of FIG. 6.

Specifically, first, in the third step, the fullerene molecule and the raw material molecule are reacted with each other, to synthesize the precursor molecule in which precursor groups are each linked to the fullerene nucleus through a spacer group. In the case where the raw material molecule is I—CF₂—SO₂F, the sulfonyl fluoride group —SO₂F is the precursor group of the proton dissociating group —SO₃H, the difluoromethylene group —CF₂— is the spacer group, and the iodine atom I is the halogen atom.

Next, in the fourth step, the precursor group —SO₂F in the precursor molecule is hydrolyzed by use of aqueous sodium hydroxide solution, to convert the precursor group into the sulfonic group sodium salt —SO₃Na, thereby obtaining the ion dissociation functional molecule. Subsequently, in the fifth step, the sodium ion in the —SO₃Na in the ion dissociation functional molecule is substituted by the hydrogen ion, to convert the ion dissociation functional molecule into the proton dissociating functional molecule, thereby obtaining poly(difluorosulfomethyl)fullerene C₆₀ shown in (E) of FIG. 6.

In Patent Documents 2 and 3, a mixed solvent of carbon disulfide: CS₂, which is a solvent capable of dissolving the fullerene therein, with hexafluorobenzene: C₆F₆, which is a solvent capable of dissolving the raw material molecule and the fluoro-type fullerene derivative used as the precursor molecule therein, is used as the reaction solvent for the third step. By use of this mixed solvent, both the solubility of the raw material system and the solubility of the reaction product system are kept high, whereby a uniform liquid-phase reaction system is maintained, without causing phase separation such as formation of a precipitate, over the period ranging from the beginning period to the ending period of the reaction. It is considered that, as a result of this, many proton dissociating groups can be introduced to the fullerene, and the proton dissociating functional molecule having a high proton conductivity can be synthesized.

The major problem in the latter steps lies in the third step. The reaction in the third step is initiated by pyrolyzing the halogen compound used as the raw material molecule and permitting the resulting halogen radicals to react with the fullerene. In the case where a compound in which a halogen atom is bonded to a fluorinated carbon chain is used as the raw material halogen compound as above-mentioned, pyrolysis of the raw material halogen compound to liberate the halogen radical needs heating to a temperature of around 200° C., even in the case where the halogen is iodine and the pyrolyzing temperature is therefore the lowest. In addition, in the cases where the halogen is other than iodine, heating to a further higher temperature is needed.

However, the boiling points of the solvents used in the above-mentioned mixed solvent are as low as 46.3° C. for carbon disulfide and 80.3° C. for hexafluorobenzene. Therefore, in order to maintain the reaction system in a liquid state at the reaction temperature around 200° C. which is higher than the boiling points of the solvents and to supply the thermal energy necessary for the progress of the pyrolytic reaction, it would be necessary to use a pressure-resistant vessel such as an autoclave as the reaction vessel and to effect the reaction at a high pressure.

The reaction at a high pressure to be conducted using an autoclave or the like would need a reaction apparatus capable of enduring high pressures and a safety equipment for securing safety, necessitating a large scale of plant and equipment investment, which is seriously disadvantageous on an industrial basis. In addition, the reaction at a high pressure is more difficult to control and is lower in working efficiency than reactions at normal pressure. Besides, since the reaction system in the third step is attended by generation of active chemical species such as the iodine radicals, there is a need for the material of the pressure-resistant vessel such as the autoclave to be high in chemical resistance (corrosion resistance) and the like. In order to endure the corrosion by the iodine radicals or the like at a high temperature of 200° C., it may be necessary to use an expensive material such as Hastelloy. Further, taking into account the progress of deterioration with time, periodic maintenance would be needed, so that a considerable maintenance cost is expected to be taken.

In consideration of the above-mentioned circumstances, it is an object of the present invention to provide a preparation method by which an ion dissociation functional molecule having a high ionic conductivity, being chemically and thermally stable under operating conditions required in an electrochemical system such as a fuel cell and being suitable for used as a material of, for example, a proton conductor in a fuel cell can be prepared in a higher yield and more easily, more efficiently, more inexpensively and more safely than by the existing art.

### DISCLOSURE OF INVENTION

Accordingly, the present invention relates to a method of preparing a raw material molecule of an ion dissociation functional molecule, including a step of reacting a reactant represented by the general formula I: AgOOC—Rf-Pre, in which a precursor (-Pre) of an ion dissociating group and a silver salt of a carboxyl group are linked to each other through an at least partly fluorinated spacer group (—Rf—), with iodine to prepare a reaction product represented by the general formula II: I—Rf-Pre,

wherein in an exhaust passage for a mixed gas of the reaction product with carbon dioxide, the mixed gas is cooled to a temperature lower than the boiling point of the reaction product and higher than the freezing point of the reaction product so as to condense the reaction product into a liquid while keeping the carbon dioxide in the gaseous state, and then

the liquefied reaction product and the mixed gas are led into a trapping vessel cooled to a temperature of not higher than the boiling point of the reaction product and not lower than the subliming point of carbon dioxide, so as to trap the reaction product.

In addition, the present invention relates also to a method of preparing an ion dissociation functional molecule, including the steps of:

synthesizing a reaction product of the general formula II by the above-mentioned method of preparing a raw material molecule of an ion dissociation functional molecule; and

synthesizing a precursor molecule represented by the general formula III: Cm(—Rf-Pre)n (where m is a natural number such that Cm can form a fullerene, and n is a natural number) by a second reaction between a fullerene molecule and the reaction product in a solvent having a boiling point of not lower than 150° C. or/and at a normal pressure or a reduced pressure, and hydrolyzing the precursor group (-Pre) of the precursor molecule so as to convert the precursor group into an ion dissociating group.

As a result of the present inventor's intensive and extensive investigations, it was found out that, in relation to the method of preparing a reaction product represented by the above-mentioned general formula II: I—Rf-Pre by a reaction between a reactant represented by the above-mentioned general formula I: AgOOC—Rf-Pre and iodine, one of the causes of the lowered yield in the existing art resides in that since carbon dioxide is generated together with the reaction product, the reaction product would be carried by the stream of carbon dioxide, and the reaction product cannot be trapped sufficiently. Based on this finding, the present inventor has devised a countermeasure against the problem, and has completed the method of preparing a raw material molecule of an ion dissociation functional molecule according to the present invention.

Specifically, in accordance with the method of preparing a raw material molecule of an ion dissociation functional molecule according to the present invention,

in an exhaust passage for a mixed gas of the reaction product with carbon dioxide, the mixed gas is cooled to a temperature lower than the boiling point of the reaction product and higher than the freezing point of the reaction product so as to condense the reaction product into a liquid while keeping the carbon dioxide in the gaseous state, and then

the liquefied reaction product and the mixed gas are led into a trapping vessel cooled to a temperature of not higher than the boiling point of the reaction product and not lower than the subliming point of carbon dioxide, so as to trap the reaction product.

Therefore, since the mixed gas can be cooled over a sufficiently long time while flowing through a long passage from the exhaust passage to the trapping vessel, it is possible to entirely cool the mixed gas to a sufficiently low temperature, to minimize the amount of the reaction product dissipating while being in a gaseous state, and, hence, to enhance the yield of the reaction product. In this case, since the temperature of the exhaust passage is kept at a temperature of lower than the boiling point of the reaction product and higher than the freezing point of the reaction product and the condensed reaction product is thereby kept in the liquid state, the liquefied reaction product can be led into the trapping vessel by providing the exhaust passage with an appropriate inclination.

On the other hand, as above-mentioned, in Patent Document 3, in selecting the reaction solvent for the synthesis of the precursor molecule represented by the general formula III: Cm(—Rf-Pre)n through the second reaction between the fullerene molecule and the above-mentioned reaction product, it was deemed as important for the reaction solvent to be a solvent high in the performance of dissolving the fullerene and the fluoro-type raw material molecule, it was considered to be necessary for the reaction solvent to be a solvent chemically stable enough not to cause a subsidiary reaction with reactive species such as radicals generated in the reaction system during the reaction, and the mixed solvent of carbon disulfide with hexafluorobenzene was selected as the reaction solvent. Since the boiling points of these component solvents are comparatively low, i.e. the boiling points are not higher than 100° C., a reaction vessel capable of enduring high pressures such as an autoclave was needed for raising the temperature to a temperature of not lower than 160° C., usually around 200° C., which is necessary for the pyrolytic reaction for generating the radicals, while keeping the solvents in a liquid state.

This point was also investigated intensively and extensively by the present inventor. As a result of the investigations, it was found out that trichlorobenzene and the like solvents are not only high in the ability to dissolve fullerenes but also higher in the ability to dissolve the halogen compound serving as the raw material molecule, than carbon disulfide and the like, and that the use of trichlorobenzene or the like as the solvent eliminates the need for the fluoro-type solvent such as hexafluorobenzene which has been used for dissolving the raw material molecule in the existing art.

Trichlorobenzene and the like solvents have boiling points of not lower than 150° C. Therefore, in the case where the above-mentioned second reaction is carried out at a temperature of not lower than 160° C., usually around 200° C., by using these solvents, the second reaction can be performed in a temperature range of not higher than the boiling points or higher than the boiling points by 10 to several tens of degrees, and the second reaction can be performed at normal pressure or in a slightly pressurized condition. Here, the “slightly pressurized condition” means a pressurized condition under which the second reaction can be carried out using an equipment not considerably different from the equipment used for reaction at normal pressure, a working efficiency not substantially different from that at normal pressure can be secured, and a production cost comparable to that in the case of normal pressure can be realized. Specifically, the “slightly pressurized condition” means a condition of being pressurized by a value in the range of 0 to 10 atm, desirably 0 to 2 atm, more desirably 0 to 1 atm, as compared with normal pressure.

Besides, in relation to the solubility of the above-mentioned precursor molecule, it has been empirically confirmed that the number of the precursor groups introduced to the precursor molecule is enhanced by use of trichlorobenzene and the like solvents, as compared with the case of using the mixed solvent of carbon disulfide and hexafluorobenzene, as will be described in Example 1 later. Thus, it has been verified that the use of trichlorobenzene and the like solvents has no problem, at least in the high-temperature condition during the reaction. Further, it has been made clear that the subsidiary reaction between the chlorine present in trichlorobenzene or the like and the radicals generated during the reaction, which has initially been a matter of concern, is not observed at all where trichlorobenzene and the like solvents are used.

From the foregoing, it has been confirmed that trichlorobenzene and the like are effective as the reaction solvent for the above-mentioned second reaction, and the present invention for synthesis of the ion dissociation functional molecule has been completed.

To be more specific, in accordance with the method of preparing an ion dissociation functional molecule according to the present invention, there are the steps of:

synthesizing a reaction product of the general formula II by the above-mentioned method of preparing a raw material molecule of an ion dissociation functional molecule; and

synthesizing a precursor molecule represented by the general formula III: Cm(—Rf-Pre)n by a second reaction between a fullerene molecule and the reaction product in a solvent having a boiling point of not lower than 150° C. or/and at a normal pressure or a reduced pressure, and hydrolyzing the precursor group (-Pre) of the precursor molecule so as to convert the precursor group into an ion dissociating group,

so that the second reaction can be carried out at normal pressure or in a slightly pressurized condition.

Therefore, the need to use a reaction vessel having a high pressure resistance such as an autoclave is eliminated, and it is possible to use a reaction vessel formed from a material being excellent in corrosion resistance though not so high in pressure resistance, such as a glass vessel. This promises a large lowering in the equipment cost needed for production equipment and maintenance thereof. In addition, the above-mentioned steps lead to enhanced working efficiency and productivity, as compared with steps at a high pressure, and, therefore, the running cost can also be lowered.

Besides, since the second reaction is carried out at normal pressure or in a slightly pressurized condition, it is possible to examine the number of the precursor groups introduced to the reaction product, by sampling the reaction mixture, during the course of the above-mentioned synthesizing process. This means that the degree of progress of the reaction can always be monitored while continuing the reaction. For example, it can be checked whether or not the number of the precursor groups introduced has reached a predetermined value, and the reaction time can be set to the required minimum value and controlled to a sufficient time. Moreover, it is easy to take various measures for controlling the second reaction, as required; for example, the variation in the concentration of the raw material molecule can be suppressed by replenishing the raw material molecule consumed in the second reaction, according to the progress of the second reaction. As a result, the quality and yield of the product are enhanced.

### BRIEF DESCRIPTION OF DRAWINGS

FIG. 1 is a flowchart of a synthesis process for a proton dissociating functional molecule, based on an embodiment of the present invention.

FIG. 2 is a schematic illustration of the configuration of a reaction apparatus used in a second step, based on the embodiment of the present invention.

FIG. 3 is a solubility curve of solubility of C₆₀ fullerene in 1,2,4-trichlorobenzene, based on the embodiment of the present invention.

FIG. 4 is a general sectional view showing the configuration of a fuel cell according to an embodiment of the present invention.

FIG. 5 (A) and (B) of FIG. 5 are examples of proton conductive fullerene derivatives shown in Patent Document 1, while (C) and (D) of FIG. 5 are examples of proton conductive fullerene derivatives shown in Patent Document 2.

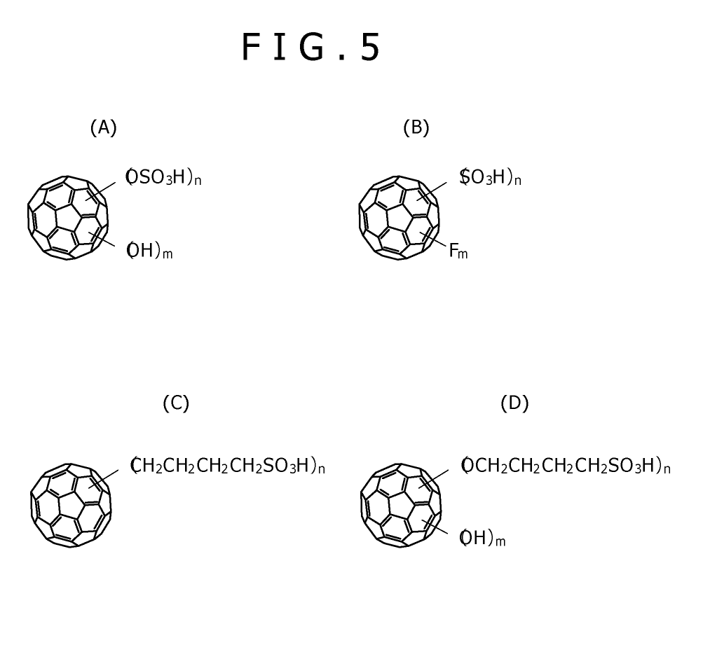

FIG. 6 (A) to (D) of FIG. 6 are examples of proton dissociating functional molecules excellent in chemical and thermal stability which are shown in Patent Document 2, and (E) of FIG. 6 is an examples of the same which is shown in Patent Document 3.

FIG. 7 shows, in the form of flowchart, a synthesis process for a proton dissociating functional molecule shown in Patent Document 3.

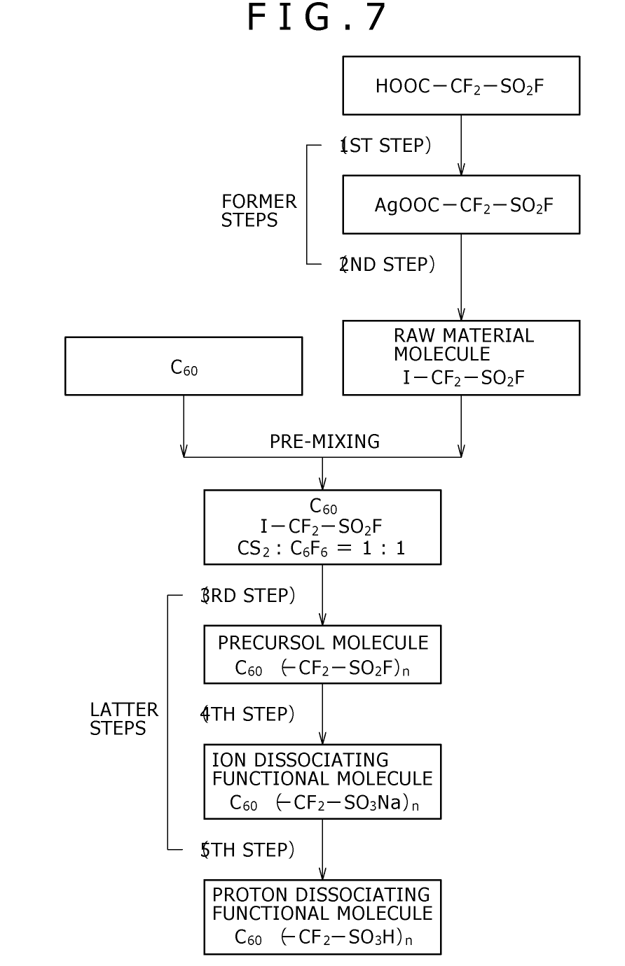

### BEST MODE FOR CARRYING OUT THE INVENTION

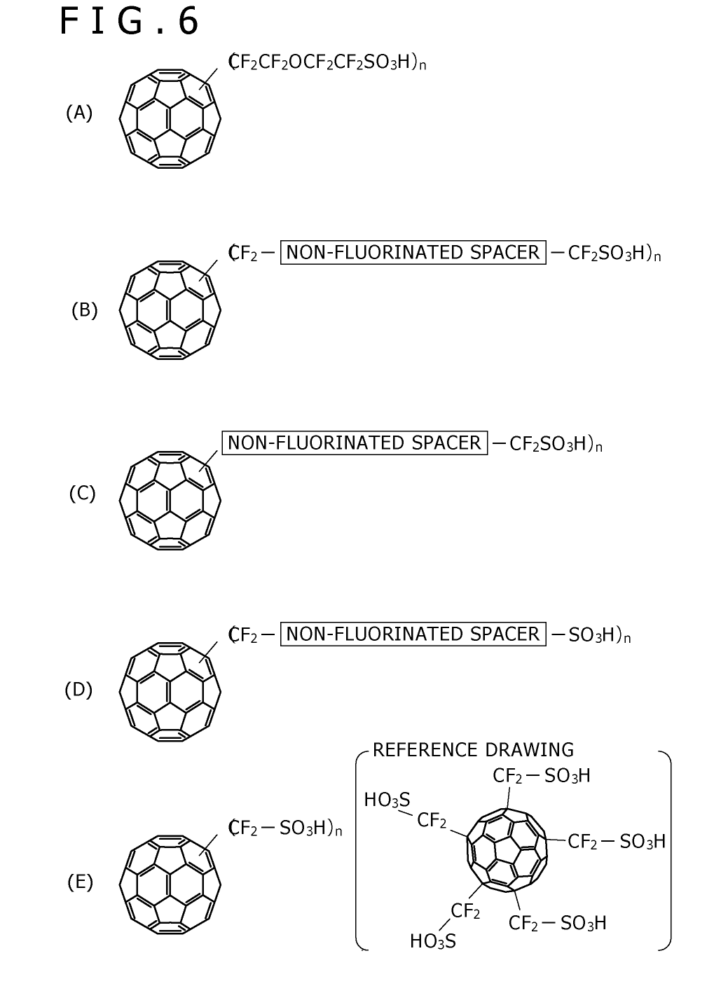

In the method of preparing a raw material molecule of an ion dissociation functional molecule based on the present invention, it is preferable to use silver(I) difluoro(fluorosulfonyl)acetate: AgOOCCF₂SO₂F as the above-mentioned reactant to thereby prepare the above-mentioned reaction product including difluoroiodomethanesulfonyl fluoride: ICF₂SO₂F. As has been mentioned above, difluoroiodomethanesulfonyl fluoride is useful as a raw material molecule for synthesis of poly(difluorosulfomethyl)fullerene C₆₀ (see (E) of FIG. 6) which has particularly excellent thermal and chemical stability and can realize high proton conductivity.

In this case, it is preferable that the reactant is reacted with iodine in equimolar relation, that the reaction is carried out at 110° C., that the above-mentioned exhaust passage is cooled to −15° C., and that the above-mentioned trapping vessel is cooled with dry ice. This makes it possible to prepare difluoroiodomethanesulfonyl fluoride in a high yield.

In the method of preparing an ion dissociation functional molecule based on the present invention, the reaction temperature for the above-mentioned second reaction is preferably 150 to 300° C. A temperature of not lower than 150° C. is preferred for pyrolysis of the iodine compound serving as the raw material molecule, and a temperature of not higher than 300° C. is needed for preventing, for example, thermal decomposition of the starting reactants and the reaction product.

A reaction solvent for the second reaction, preferably, includes a halobenzene, specifically at least one solvent selected from the following group, in which the parenthesized numerical values affixed to the solvent names show the boiling points of the solvents at normal pressure (1 atm).

Group of Solvents:

1,2,4-trichlorobenzene (210° C.), 1,2,3-trichlorobenzene (218 to 219° C.), n-propylbenzene (159° C.), cumene (isopropylbenzene) (153° C.), n-butylbenzene (183° C.), iso-butylbenzene (173° C.), sec-butylbenzene (173 to 174° C.), tert-butylbenzene (168° C.), o-dibromobenzene (224° C.), m-dibromobenzene (219.5° C.), p-dibromobenzene (218 to 219° C.), o-dichlorobenzene (180 to 183° C.), m-dichlorobenzene (172° C.), p-dichlorobenzene (174° C.), 1-phenylnaphthalene (334° C.), and 1-chloronaphthalene (263° C.).

As has been described above, these solvents are not only high in the ability to dissolve fullerenes but also high in the ability to dissolve the halogenated compounds which are the raw material molecules. The solvent may be selected in correspondence with the reaction temperature for the second reaction so that the second reaction is conducted at a temperature of not higher than the boiling point of the solvent or of higher than the boiling point by a value in a range of 10 to several tens of degree, whereby the second reaction can be performed at normal pressure or in a slightly pressurized condition.

In addition, the reaction solvent for the second reaction may be a single solvent or a mixed solvent. A single solvent has the merit of simple working, whereas a mixed solvent has the merit that it can realize such characteristics that cannot be realized with a single solvent. For example, the reaction solvent for the second reaction preferably includes 1,2,4-trichlorobenzene used as a single solvent.

Alternately, a mixed solvent of trichlorobenzene and hexafluorobenzene in a volume ratio of 1:1 is preferably used as the reaction solvent for the second reaction. This, as compared with the case of using a mixed solvent of carbon disulfide and hexafluorobenzene, is advantageous in that the use of the toxic carbon disulfide can be obviated, the pressure of the reaction system can be set lower, so that safety and working efficiency are enhanced, and a reduction in cost can be achieved.

Besides, preferably, the above-mentioned raw material molecule is slowly added dropwise to the solution of the fullerene molecule in the reaction solvent for the second reaction, according to the progress of the second reaction. According to the methods described in Patent Documents 2 and 3 in which the whole amount of the raw material molecule is first supplied into the reaction system, the concentration of the raw material molecule is maximum at the start of the reaction, and is thereafter monotonously decreased as the second reaction progresses, so that in the finishing period of the reaction, the raw material molecule present in the beginning period has been mostly consumed. Thus, the concentration of the raw material molecule varies largely during the second reaction. On the other hand, the slow dropwise addition ensures that the raw material molecule lost according to the progress of the second reaction is replenished, whereby the variation in the concentration of the raw material molecule during the second reaction can be suppressed, the second reaction can be performed stably under the reduced-variation conditions; for example, a dimerizing reaction between the raw material molecules can be restrained as securely as possible. In addition, since the concentration of the raw material molecule can be set by far lower than the initial concentrations in the methods of Patent Documents 2 and 3, it is possible to use a solvent lower in the ability to dissolve the raw material molecule than the solvents used in the methods of Patent Documents 2 and 3.

In this case, preferably, stirring is continued even after the dropwise addition so as to effect the second reaction. The completion of the dropwise addition does not means the completion of the second reaction, and, therefore, it is preferable to continue the stirring even after the dropwise addition so that as much as possible of the fullerene is brought into the second reaction.

As the fullerene molecule in the present invention, any of known fullerene molecules can be used. Examples of the fullerene molecule which can be used here include C₃₆, C₆₀, C₇₀, C₇₆, C₇₈, C₈₂, C₈₄, C₉₀, C₉₆, and C₂₆₆. The fullerene molecule may be a molecule obtained, or as if obtained, through losing a part of a spherical carbon molecule like C₃₆.

In the fullerene preparation methods which are used at present, C₆₀ and C₇₀ or a mixture thereof can be used particularly preferably, since the ratios of formation of C₆₀ and C₇₀ are overwhelmingly high, so that use of C₆₀ and/or C₇₀ is advantageous on a production cost basis, and, in general, the reactivity of fullerene molecules decreases with an increase in the size of the fullerene molecules. The fullerene molecules each have a uniform shape irrespective of the direction in which proton carriers migrate, so that the use of the fullerene molecules makes it possible to attain an enhanced proton mobility and to obtain a high proton conductivity performance.

As a reaction vessel for the second reaction, a glass-made vessel is preferably used. Alternately, a vessel having a metallic surface lined with a glass layer is preferably used as the reaction vessel for the second reaction. Glasses are materials excellent in corrosion resistance, and are inexpensive, though not so high in pressure resistance. Therefore, glass-made reaction vessels are optimum for use in the synthesis process which, according to the present invention, can be carried out at normal pressure or in a slightly pressurized condition.

The preparation method, preferably, further includes the step of replacing the ion bonded to the ion dissociating group formed in the above-mentioned hydrolyzing step with a predetermined ion so as to obtain a predetermined ion dissociation functional molecule. The hydrolyzing step is preferably carried out in a basic environment and, as a result, an alkaline metal ion such as sodium ion is in many cases being bonded to the ion dissociating group formed upon the hydrolyzing step. Therefore, by replacing the alkali metal ion or the like with a desired ion, for example, hydrogen ion, the ion dissociation functional molecule containing the desired ion can be obtained.

In this manner, a proton dissociating functional molecule is preferably obtained as the ion dissociation functional molecule. Proton dissociating functional molecules are useful as a material of, for example, a proton conduction membrane used in a fuel cell.

The ion dissociating group is preferably a proton dissociating group selected from the group consisting of the hydrogen sulfate ester group —OSO₂OH, the sulfonic group —SO₂OH, the dihydrogenphosphoric ester group —OPO(OH)₂, the monohydrogenphosphoric ester group —OPO(OH)—, the phosphono group —PO(OH)₂, the carboxyl group —COOH, the sulfonamide group —SO₂—NH₂, the sulfonimide group —SO₂—NH—SO₂—, the methanedisulfonyl group —SO₂—CH₂—SO₂—, the carboxamide group —CO—NH₂, and the carboximide group —CO—NH—CO—. The hydrogen contained in these functional groups is liable to be liberated as proton, and, therefore, these functional groups are excellent proton dissociating functional groups.

Each of these functional groups is a proton dissociating group when in the above-mentioned condition. In the condition where the hydrogen ion in the functional group has been replaced with another cation, the functional group functions as an ion dissociating group capable of dissociating the another cation. The another cation is preferably a cation of an alkali metal atom or the like, specific examples of which include lithium ion, sodium ion, potassium ion, rubidium ion, and cesium ion.

Now, some preferred embodiments of the present invention will be described specifically below, referring to the drawings.

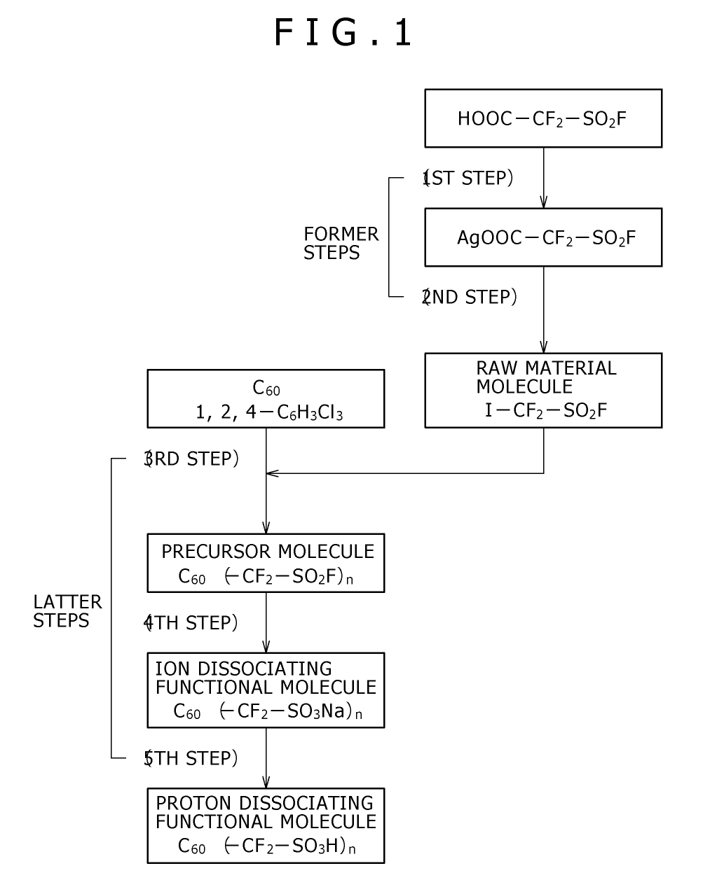

FIG. 1 is a flowchart of a synthesis process for an ion dissociation functional molecule based on an embodiment of the present invention. FIG. 1 shows an example in which the fullerene molecule is C₆₀ and the raw material molecule to be brought into reaction with the fullerene molecule is difluoroiodomethanesulfonyl fluoride: ICF₂SO₂F.

In the flow of synthesis shown in FIG. 1, first, in the first and second steps in the former steps, the raw material molecule ICF₂SO₂F is synthesized (refer to Patent Document 3 and Non-patent Document 1).

In the first step, silver difluoro(fluorosulfonyl)acetate AgOOCCF₂SO₂F is synthesized from difluoro(fluorosulfonyl)acetic acid HOOCCF₂SO₂F by the following reaction.

Ag₂CO₃+2HOOCCF₂SO₂F→H₂O+CO₂+2AgOOCCF₂SO₂F

While this reaction has hitherto been known as a high-yield reaction, the yield can further be enhanced by optimizing the reaction temperature and the dropping rate in dropwise addition of difluoro(fluorosulfonyl)acetic acid to an ether solution of silver carbonate, as will be described in Example 1 later.

Next, in the second step, difluoroiodomethanesulfonyl fluoride: ICF₂SO₂F is synthesized by permitting iodine to act on silver difluoro(fluorosulfonyl)acetate: AgOOCCF₂SO₂F by the following reaction.

AgOOCCF₂SO₂F+I₂→ICF₂SO₂F+CO₂+AgI

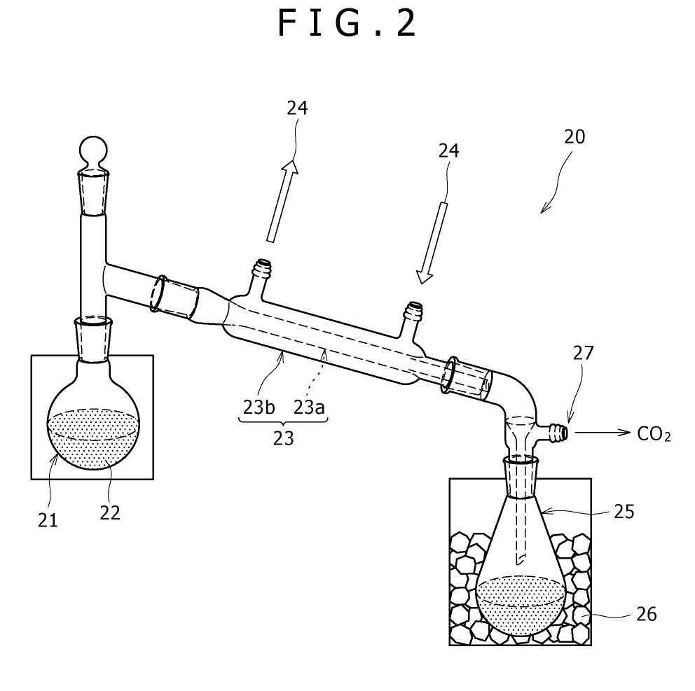

FIG. 2 is a schematic illustration of the configuration of a reaction apparatus 20 used in the second step. A reaction vessel 21 is equipped with a cooling pipe 23 as the above-mentioned exhaust passage for enabling direct distillation of a reaction mixture 22 in the reaction vessel 21. The cooling pipe 23 is a double pipe, and a cooling liquid 24 cooled to a predetermined temperature is allowed to flow between an inner pipe 23a and an outer pipe 23b, whereby a gas in the inner pipe 23a can be cooled to a predetermined temperature. A trap 25 is provided substantially in connection with the cooling pipe 23, and cooling with a coolant such as dry ice and liquid nitrogen is conducted therein, whereby substances being gaseous or showing a comparatively high vapor pressure at normal temperature can be condensed, to be trapped (collected) as liquid or solid.

In the second step, the cooling liquid 24 is cooled to a temperature, for example −15° C., which is lower than the boiling point (supposed to be about 40° C.) of difluoroiodomethanesulfonyl fluoride as the reaction product and higher than the freezing point of the fluoride, whereby part of the reaction product is condensed into a liquid while keeping carbon dioxide in a gaseous state. The trap 25 is cooled with dry ice 26 to a temperature, for example about −78° C., which is not higher than the boiling point of difluoroiodomethanesulfonyl fluoride and not lower than the subliming point of carbon dioxide.

As a result, the mixed gas of difluoroiodomethanesulfonyl fluoride and carbon dioxide generated in the reaction vessel 21 can be cooled over a sufficiently long time while passing through the long passage from the cooling pipe 23 to the trap 25. Therefore, it is possible to entirely cool the mixed gas to a sufficiently low temperature, to minimize the amount of difluoroiodomethanesulfonyl fluoride dissipating as a gas, and to enhance the yield of the desired product. In this instance, since the temperature of the cooling pipe 23 is kept at a temperature of lower than the boiling point of difluoroiodomethanesulfonyl fluoride and higher than the freezing point of the fluoride, the condensed difluoroiodomethanesulfonyl fluoride is kept in a liquid state, and is led by the inclination of the cooling pipe 23 into the trap 25.

Besides, further improvements lies in the following two points:

<1> Since difluoroiodomethanesulfonyl fluoride as the reaction product is adsorbed on iodine, silver difluoro(fluorosulfonyl)acetate and iodine are added in a molar ration of 1:1 so as to reduce the amount of the iodine which is left unreacted upon the reaction.

<2> In order that the reaction is accelerated and is finished in a short period, the temperature of the reaction mixture 22 is raised from 100° C. adopted in the existing art to 110° C. This can reduce the amount of the reaction product which is once trapped but is lost through re-evaporation, which is advantageous in trapping the reaction product having high volatility.

In addition, in order to reduce the proportion of the matter lost through adhesion to walls or the like, the amount of the sample treated in one run of the step is increased. By these contrivances, the yield was enhanced successfully, to realize a yield of 65% as will be described in Example 1 later.

Subsequently, in the third to fifth steps in the latter steps, difluoroiodemethanesulfonyl fluoride: ICF₂SO₂F as the raw material molecule is let act on the fullerene C₆₀, and then the resulting precursor molecule is hydrolyzed, to synthesize poly(difluorosulfomethyl)fullerene C₆₀ shown in (E) of FIG. 6 as the proton dissociating functional molecule.

First, in the third step, the fullerene molecule and the raw material molecule are reacted with each other, to synthesize the precursor molecule in which a precursor group is linked to the fullerene nucleus through a spacer group. In the case where the raw material molecule is I—CF₂—SO₂F, the sulfonyl fluoride group —SO₂F is the precursor group of the proton dissociating group —SO₃H, the perfluoromethylene group —CF₂— is the spacer group, and the iodine atom I is the halogen atom.

Next, in the fourth step, the precursor group —SO₂F in the precursor molecule is hydrolyzed by use of an aqueous sodium hydroxide solution, whereby the precursor group —SO₂F is converted into a sulfonic group sodium salt —SO₃Na, to obtain the ion dissociation functional molecule. Subsequently, in the fifth step, the sodium ion of —SO₃Na in the ion dissociation functional molecule is replaced with the hydrogen ion, to obtain poly(difluorosulfomethyl)fullerene C₆₀ shown in (E) of FIG. 6 as the proton dissociating functional molecule.

In Patent Documents 2 and 3, the mixed solvent of hexafluorobenzene having a low boiling point and carbon disulfide was used as the reaction solvent for the third step. Therefore, the pressure was brought to a high pressure at the reaction temperature of around 200° C., and it was necessary to carry out the reaction in a pressure-resistant vessel such as an autoclave. On the other hand, in this embodiment of the present invention, since a solvent having a boiling point of not lower than 150° C., for example, 1,2,4-trichlorobenzene with a boiling point of 210° C. is used, so that the reaction can be carried out at normal pressure or in a slightly pressurized condition at a reaction temperature of around 200° C. As a result of the reaction being carried out at normal pressure or in a slightly pressurized condition, various merits are produced.

For example, use can be made of a glass-made reaction vessel which is excellent in corrosion resistance and is inexpensive, though not so high in pressure resistance. Therefore, it is possible to remarkably lower the plant and equipment cost needed for the production equipment and maintenance thereof. Besides, as compared with steps carried out at a high pressure, the working efficiency and productivity are enhanced, so that the running cost can also be lowered.

In addition, as will be described later in Example 1, it is possible to sample the reaction mixture and to constantly monitor the degree of progress of the reaction while continuing the reaction, and it is easy to take various measures for controlling the reaction, as required.

In addition, the raw material molecule can be slowly added dropwise to the solution of the fullerene molecule, according to the progress of the reaction, instead of being wholly added to the solution in the beginning of the reaction. In the method in which the whole amount of the raw material molecule is added to the reaction system in the beginning of the reaction, the concentration of the raw material molecule is maximum at the start of the reaction, is then monotonously decreased with the progress of the reaction, and, therefore, the concentration of the raw material molecule varies largely during the synthesizing process. On the other hand, where the raw material molecule is slowly added dropwise as above-mentioned, the raw material molecule lost in the reaction is replenished according to the progress of the reaction, whereby the variation in the concentration of the raw material molecule in the reaction mixture during the synthesizing process can be suppressed, and the reaction can be effected stably and efficiently in the vicinity of the optimum concentration of the raw material molecule. Besides, the concentration of the raw material molecule in this case can be set by far lower than the initial concentrations in the methods in which the whole amount of the raw material molecule is added to the reaction system in the beginning, which makes it possible to use any of a diversity of solvents lower in the ability to dissolve the raw material molecule, as compared with the solvents used in the method of Patent Document 2 or 3.

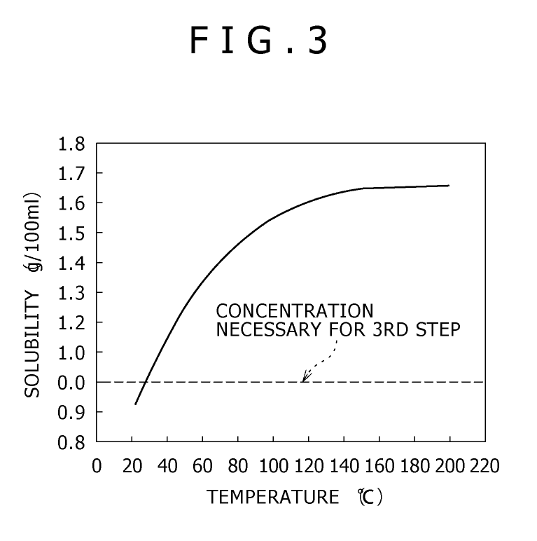

FIG. 3 is a solubility curve of solution of C₆₀ fullerene in 1,2,4-trichlorobenzene. The solubility is represented in terms of the number of grams of C₆₀ fullerene capable of being dissolved in 100 ml of 1,2,4-trichlorobenzene. The solubility of C₆₀ fullerene is comparatively low at normal temperature, but increases with a rise in temperature, to reach a sufficiently high value at the reaction temperature of around 200° C. (150 to 240° C.).

### EXAMPLES

Now, by showing preferred embodiments (Examples) of the present invention, the method of preparing an ion dissociation functional molecule based on the present invention will be described specifically below, together with the analytical results of proton dissociating functional molecules prepared as ion dissociation functional molecules by the method, and with the measurement results of proton conductivity of the proton conductors including the proton dissociating functional molecules.

Examples 1 to 3 are examples in which proton dissociating functional molecules were synthesized according to the flow of the synthetic process shown in FIG. 1. The reaction in the third step, which is the above-mentioned second reaction, was carried out at normal pressure in a glass vessel by using 1,2,4-trichlorobenzene singly as the reaction solvent.

### Example 1

Example 1 is an example in which the third step was carried out at a reaction temperature of 160° C. over a reaction time of four days.

The First Step:

In the first step, silver

difluoro(fluorosuofonyl)acetate AgOOCCF₂SO₂F was synthesized from difluoro(fluorosulfonyl)acetic acid HOOCCF₂SO₂F by the following reaction. In this example, the temperature and the dropping rate of difluoro(fluorosulfonyl)acetic acid at the time of reaction were optimized, to enhance the yield as compared with that in the existing art. As the conditions for the optimization, a reaction temperature of 15° C. and a dropping time (dropwise addition time) of 20 min are recommendable.

Ag₂CO₃+2HOOCCF₂SO₂F→H₂O+CO₂+2AgOOCCF₂SO₂F

By use of a thermostatic bath, 50 g (182 mmol) of silver carbonate was dispersed in diethyl ether at 15° C., and, while stirring the dispersion, 65 g (363 mmol) of difluoro(fluorosulfonyl)acetic acid HOOCCF₂SO₂F was slowly added dropwise to the dispersion. The dropping time was 20 min. After the dropwise addition, stirring of the reaction mixture was continued at the temperature of 15° C. for about one day, to effect the reaction. After the reaction was over, the reaction mixture was filtered to remove the unreacted silver carbonate, and the precipitate was washed three times with diethyl ether. Subsequently, the ether was evaporated off, to obtain a white solid matter. The solid matter was recrystallized from a mixed solvent of diethyl ether and hexane, to obtain white needle-like crystals of pure silver difluoro(fluorosulfonyl)acetate. The desired product was obtained in an amount of 98.5 g, the yield being 96%. Identification of the product was carried out by the IR (infrared spectroscopic) method.

### Comparative Example 1

Dropwise addition of difluoro(fluorosulfonyl)acetic acid and the subsequent stirring were carried out at room temperature, to effect the reaction at room temperature. The other conditions were the same as in Example 1.

The desired product was obtained in an amount of 96.4 g, the yield being 94%. Generation of heat at the time of the reaction raises the actual reaction temperature to 30° C. or above, and the too vigorous reaction leads to a heterogeneous reaction, whereby the unreacted silver carbonate is left and the yield of the desired product is slightly lowered.

### Comparative Example 2

By use of a thermostatic bath, dropwise addition of the reactant and stirring of the reaction mixture were carried out at a temperature of 5° C., to effect the reaction at the temperature of 5° C. The other conditions were the same as in Example 1.

The desired product was obtained in an amount of 92.5 g, the yield being 90.1%. When the temperature at the time of reaction is too low, the reaction proceeds insufficiently, so that the unreacted silver carbonate is left and the yield of the desired product is lowered.

### Comparative Example 3

Dropwise addition of the reactant and stirring of the reaction mixture were carried out at room temperature, to effect reaction at room temperature. The dropping time was 2 min. The other conditions were the same as in Example 1.

The desired product was obtained in an amount of 89.2 g, the yield being 87%. The reasons for the lower yield than that in Comparative Example 1 are as follows. Due to the generation of heat at the time of the reaction, the actual reaction temperature is 30° C. or above, leading to too vigorous a reaction. In addition, the high dropping rate renders the reaction more vigorous, whereby the reaction becomes further heterogeneous, and the amount of silver carbonate left unreacted is increased.

### Comparative Example 4

Dropwise addition of the reactant and stirring of the reaction mixture were carried out at room temperature, to effect reaction at room temperature. The dropping time was 60 min. The other conditions were the same as in Example 1.

The desired product was obtained in an amount of 91.3 g, the yield being 89%. The reason for the lower yield than that in Comparative Example 1 are as follows. Due to the too low dropping rate, the concentration of difluoro(fluorosulfonyl)acetic acid at the time of reaction is too low, so that the reaction proceeds insufficiently, silver carbonate is partly left unreacted, and the yield of the desired product is lowered.

The Second Step:

In the second step, difluoroiodomethanesulfonyl fluoride ICF₂SO₂F was synthesized by letting iodine act on silver difluoro(fluorosulfonyl)acetate AgOOCCF₂SO₂F by the following reaction.

AgOOCCF₂SO₂F+I₂→ICF₂SO₂F+CO₂+AgI

As shown in FIG. 2, a reaction apparatus equipped with a cooling pipe for enabling direct distillation of a reaction mixture in a reaction vessel was assembled. The reaction vessel was charged with 30 g (105 mmol) of silver difluoro(fluorosulfonyl)acetate and 26.7 g (105 mmol) of iodine, followed by stirring the mixture wall. Then, the temperature is slowly raised from room temperature to 110° C. over a period of about 20 min, and the reaction system was kept at the fixed temperature of 110° C. In this case, the temperature of the cooling pipe was kept at −15° C. by circulating a cooled Nybrine Coolant (tradename of a cooling liquid, produced by Maruzen Chemical Corporation), while the trap was cooled with dry ice to keep a temperature of about −78° C.

Due to heating, the mixed gas of difluoroiodomethanesulfonyl fluoride (estimated boiling point: about 40° C.) and carbon dioxide produced upon the reaction is caused to flow out of the reaction vessel through the cooling pipe. In view of this, only difluoroiodomethanesulfonyl fluoride was selectively condensed by the above-mentioned cooling means, and was trapped by the trapping vessel, separately from the carbon dioxide gas.

Difluoroiodomethanesulfonyl fluoride was obtained in an amount of 17.7 g, the yield being 65%. Thus, the yield was enhanced from 48%, which is the value described in Patent Document 3, to 65%. Identification of the final product was carried out by the IR method, the ¹³C-NMR (nuclear magnetic resonance) method and the ¹⁹F-NMR method, whereby the product was confirmed to be the same substance as obtained in the previous patent application.

### Modified Example 1

In the same manner as in Patent Document 3, iodine was added in an amount (157 mmol) of 1.5 times the equivalent, and the heating temperature was set at 100° C. Incidentally, the temperature of the cooling pipe in the distillation apparatus was set to −15° C., and difluoroiodomethanesulfonyl fluoride was trapped by use of an ice bath.

The difluoroiodomethanesulfonyl fluoride was obtained in an amount of 14.2 g, the yield being 52%. The reason for the improvement in yield as compared with Patent Document 3 is considered to be as follows. The cooling of the cooling pipe from room temperature to −15° C. might cause the mixed gas to be cooled more effectively, leading to better separation between difluoroiodomethanesulfonyl fluoride and the carbon dioxide gas.

### Modified Example 2

While the heating temperature was kept at 100° C., addition of an excess of iodine was obviated, the temperature of the cooling pipe was set to −15° C., and difluoroiodomethanesulfonyl fluoride was trapped by use of an ice bath.

The difluoroiodomethanesulfonyl fluoride was obtained in an amount of 15.0 g, the yield being 55.1%. The reason for the enhanced yield as compared with Modified Example 1 lies in that the addition of an excess of iodine was obviated and, therefore, the loss of difluoroiodomethanesulfonyl fluoride due to adsorption thereof to iodine was less.

### Modified Example 3

Addition of an excess of iodine was obviated, the heating temperature was set to 110° C., the temperature of the cooling pipe was set to −15° C., and difluoroiodomethanesulfonyl fluoride was trapped by use of an ice bath.

The difluoroiodomethanesulfonyl fluoride was obtained in an amount of 15.96 g, the yield being 58.6%. The reason for the enhanced yield as compared with Modified Example 2 lies in that, as has been described in the embodiment above, the raised temperature led to an enhanced reaction rate, whereby the reaction was finished in a shorter time, which is advantageous in trapping the reaction product having high volatility.

### Comparative Example 5

While addition of an excess of iodine was obviated and the heating temperature was set to 110° C., but use was made of an apparatus in which difluoroiodomethanesulfonyl fluoride is caused to flow out into the trapping vessel without passing through a cooling pipe of a distillation apparatus, and the difluoroiodomethanesulfonyl fluoride was trapped by use of an ice bath.

The difluoroiodomethanesulfonyl fluoride was obtained in an amount of 9.8 g, the yield being 36%. The reason for the lowered yield as compared with Example 3 lies in that the omission of the cooling pipe led to insufficient cooling of the mixed gas, whereby the amount of difluoroiodomethanesulfonyl fluoride which was discharged into the atmosphere in a gaseous state together with carbon dioxide and which could not therefore be trapped (collected) was increased.

### Comparative Example 6

While addition of an excess of iodine was obviated and the heating temperature was set to 110° C., use was made of an apparatus in which difluoroiodomethanesulfonyl fluoride is caused to flow out into the trapping vessel without passing through a cooling pipe of a distillation apparatus, and the difluoroiodomethanesulfonyl fluoride was trapped by use of a trapping vessel cooled with liquid nitrogen.

The difluoroiodomethanesulfonyl fluoride was obtained in an amount of 6.3 g, the yield being 23%. The reason for the lowered yield as compared with Comparative Example 5 lies in that difluoroiodomethanesulfonyl fluoride was trapped by the trapping vessel at the liquid nitrogen temperature together with carbon dioxide, and, in returning from the liquid nitrogen temperature to room temperature, much of the difluoroiodomethanesulfonyl fluoride was carried away by the carbon dioxide turned into the gaseous state, resulting in a loss of the intended product.

### Comparative Example 7

While addition of an excess of iodine was obviated and the heating temperature was set to 110° C., use was made of an apparatus in which difluoroiodomethanesulfonyl fluoride is caused to flow out into the trapping vessel without passing through a cooling pipe of a distillation apparatus, and the difluoroiodomethanesulfonyl fluoride was trapped by use of dry ice.

The difluoroiodomethanesulfonyl fluoride was obtained in an amount of 11.4 g, the yield being 42%. Since the desired product was trapped by use of dry ice in place of the ice bath, the yield was enhanced as compared with Comparative Example 5, but was still lower than that in Modified Example 3. The reason lies in that the omission of the cooling pipe led to insufficient cooling of the mixed gas, resulting in an increase in the amount of the difluoroiodomethanesulfonyl fluoride which was discharged into the atmosphere in the gaseous state together with carbon dioxide and which could not therefore be trapped (collected).

The Third Step:

In the third step, fullerene C₆₀ and the raw material molecule ICF₂SO₂F obtained in the second step were reacted by the following reaction, to introduce the sulfonyl fluoride groups to the fullerene, thereby obtaining the above-mentioned precursor molecule represented by the general formula: C₆₀(—CF₂—SO₂F)ₙ (where n is about 11 on average) (this includes a plurality of reaction products differing in the number n of the functional groups introduced and in the positions of introduction, here and hereinafter).

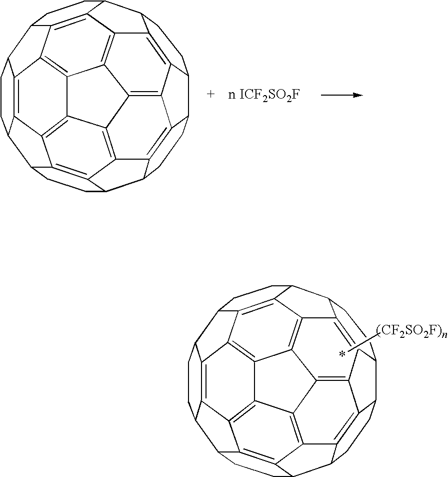

First, a 200 ml three-necked glass flask was equipped with a dropping funnel, a condenser, a thermometer and a stirrer. In a nitrogen gas stream atmosphere, 1.0 g of C₆₀ fullerene was transferred into the three-necked flask, and 100 ml of 1,2,4-trichlorobenzene was added thereto. While stirring the admixture and maintaining the reaction temperature at 160° C., 8.7 g (24 equivalents in relation to the fullerene) of a raw material molecule ICF₂SO₂F divided into three portions was added dropwise through the dropping funnel over an addition time of three days, in the manner of adding one portion per day. Such a slow addition of the raw material molecule makes it possible to effect the reaction under the condition where the concentration of the raw material molecule is kept substantially constant. After the dropwise addition was over, the reaction was carried out with stirring at 160° C. for four days. Since the reaction proceeds comparatively slowly, this level of time is needed to obtain a desired number of the functional groups added, per fullerene molecule.

After the reaction was over, the reaction mixture was cooled, and then 1,2,4-trichlorobenzene was distilled off at 100° C. and a reduced pressure from the reaction mixture, followed by vacuum drying at 100° C., to obtain 3.03 g of a reaction product including the precursor molecule in the form of a dark brown powder.

In the above-mentioned reaction, the raw material molecule is pyrolyzed at an end portion on the iodine atom side into the iodine atom and a radical (—CF₂—SO₂F), and the resulting radicals are each added to the fullerene molecule through the unpaired electron which has been bound to the iodine atom. Each sulfonyl fluoride groups —SO₂F thus introduced as the precursor group to the fullerene is hydrolyzed in the subsequent reaction step, thereby being converted into the sulfonic group.

According to the Fourier transform infrared absorption method (FT-IR), not any absorption by unreacted fullerene was observed. In addition, upon measurement by the time-of-flight mass spectrometry (TOF-MS), also, not any peak at 720 corresponding to unreacted fullerene was observed. These results show that substantially 100% of the fullerene was reacted. Besides, it was made clear from the TOF-MS spectrum that the precursor molecule is a compound in which an average of eleven difluoro(fluorosulfonyl) group —CF₂—SO₂F are bonded to the C₆₀ fullerene.

The Fourth Step:

In the fourth step, the above-mentioned precursor molecule was reacted with an aqueous alkali solution such as aqueous sodium hydroxide (NaOH) solution and aqueous potassium hydroxide (KOH) solution to hydrolyze the sulfonyl fluoride groups —SO₃F as illustrated below, thereby obtaining an ion dissociation functional molecule in which the sulfonic group sodium salts are each linked to the C₆₀ fullerene through the spacer group —CF₂—.

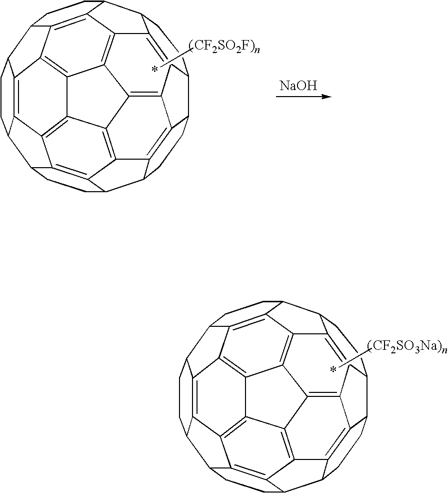

The reaction liquid used in this step is preferably configured by adding THF (tetrahydrofuran) to the aqueous sodium hydroxide solution for hydrolysis of the precursor molecule. For example, 0.2 g of the precursor molecule is dissolved in 20 ml of THF, and 10 ml of 1 M aqueous sodium hydroxide solution is added to the solution, followed by stirring to effect reaction.

After the reaction was over, the excess of sodium hydroxide was separated from the above-mentioned aqueous solution phase by silica gel column chromatography using a mixed solvent of water and THF as an eluent, whereby a purified ion dissociation functional molecule could be obtained.

Since the dried precursor molecule is dissolved in water with difficulty, it is preferable, for dissolving the precursor molecule to obtain a solution, to add THF as a solvent to the reaction liquid.

Sodium hydroxide in an amount of 1 mol is needed for hydrolysis of 1 mol of the sulfonyl fluoride group —SO₂F. Therefore, where the number of the spacered proton conductive functional group precursors introduced per one fullerene molecule is 11, the minimum mass amount of sodium hydroxide necessary for hydrolyzing the whole amount of the sulfonyl fluoride groups introduced to the fullerene molecules is 11 times the mass amount of the fullerene (namely, the amount of sodium hydroxide is 11 equivalents at minimum per 1 equivalent of fullerene). Normally, the hydrolysis is conducted in the presence of sodium hydroxide in excess of this minimum amount so that the whole amount of the sulfonyl fluoride groups can be hydrolyzed.

The aqueous sodium hydroxide solution phase upon the hydrolytic reaction contains by-products and an excess of sodium hydroxide, in addition to the desired ion dissociation functional molecule. In recovering the ion dissociation functional molecule from this aqueous solution by the above-mentioned silica gel column chromatography, the above-mentioned mixed solvent of water and THF is preferably used as an eluent, for enhancing the effect on removal of sodium hydroxide. When water is used singly as the eluent, the strong polarity of the eluent would cause the sodium hydroxide once adsorbed onto the silica gel to be gradually released, so that sodium hydroxide is mixed into the eluate. On the other hand, when the polarity of the eluent is lowered by addition of THF, the sodium hydroxide adsorbed onto the silica gel is kept adsorbed, so that mixing of sodium hydroxide into the eluate does not occur.

In this manner, it is possible to obtain a neutral solution containing only the ion dissociation functional molecule which is extremely highly soluble in water. At this time point, the solvent (THF and water) is preferably removed from the eluate. Removal of the solvent is preferably conducted through evaporating off the solvent from the eluate at a reduced pressure by an evaporator.

The Fifth Step:

In the fifth step, protonation of the sulfonic group alkali salts in the ion dissociation functional molecules was conducted, to obtain a proton dissociating functional molecule in which the sulfonic groups are each linked to the C₆₀ fullerene through the spacer group —CF₂—.

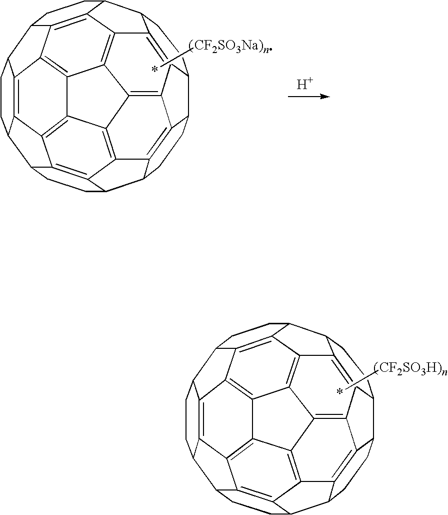

Specifically, after the removal of the solvent (water and THF) from the reaction product including only the ion dissociation functional molecule, an aqueous solution of the ion dissociation functional molecule was prepared, and the solution was supplied to a cation exchange resin column substituted by the hydrogen ion. In this case, the sodium ions Na⁺ of the ion dissociation functional molecule were replaced with the hydrogen ions H⁺ in the column, whereby the proton dissociating functional molecule could be obtained in the eluate (effluent).

Incidentally, the protonation can be effected not only by use of the cation exchange resin but also by use of an inorganic strong acid such as HCl, H₂SO₄, HClO₄, and HNO₃. Alternatively, other arbitrary preferable methods may also be used for the protonation.

(Evaluation of Proton Conductivity)

A sample of the proton dissociating functional molecule synthesized as above-described was vacuum dried at room temperature for 12 hours, and then the resulting powder was molded by a tablet molding machine into a pellet having a thickness of about 300 μm. When the powder is sandwiched between gold electrodes at the time of palletizing, a pellet is obtained in the state of being sandwiched between the gold electrodes upon the pressure molding.

The pelletized sample was analyzed by an impedance analyzer, and, from the measurement data, a proton conductivity in dry state of 2.9×10⁻³ Scm⁻¹ was obtained. Here, the proton conductivity in dry state means the proton conductivity measured for the pelletized sample in vacuum created by evacuation with a rotary pump.

### Example 2

Example 2 is an example in which the reaction in the third step was carried out at a reaction temperature of 160° C. over a reaction time of seven days. The other synthesizing steps were the same as in Example 1.

From 1 g of fullerene, 3.1 g of the precursor molecule was obtained. The number of the proton dissociating groups introduced per one fullerene molecule was 12 on average, and the proton conductivity in dry state was 5.6×10⁻³ Scm⁻¹.

### Example 3

Example 3 is an example in which the reaction in the third step was carried out at a reaction temperature of 160° C. over a reaction time of 10 days. The other synthesizing steps were the same as in Example 1.

From 1 g of fullerene, 3.2 g of the precursor molecule was obtained. The number of the proton dissociating groups introduced per one fullerene molecule was 12 on average, and the proton conductivity in dry state was 5.8×10⁻³ Scm⁻¹. The proton conductivity was substantially the same as in Example 2, probably because the numbers of the proton dissociating groups introduced per one fullerene molecule in the two examples were substantially the same.

In the cases of Examples 1 to 3, the preferable reaction time at a reaction temperature of 160° C. was about four to ten days.

### Examples 4 to 6

Examples 4 to 6 are examples in which proton dissociating functional molecules were synthesized according to the flow of the synthetic process shown in FIG. 1. The reaction in the third step, which is the above-mentioned second reaction, was carried out at a pressure of about 10 atm in a pressure resistant vessel by using as a reaction solvent a mixed solvent of trichlorobenzene and hexafluorobenzene in a volume ratio of 1:1. The other conditions are the same as in Example 1.

Carbon disulfide: CS₂ used in Patent Documents 2 and 3 is strongly toxic and is flammable, so that it has problems from the viewpoint of mass production. In addition, since the boiling point of carbon disulfide is as low as 46° C., putting carbon disulfide into reaction in an autoclave, the pressure inside the autoclave will be raised to 30 atm, which is highly dangerous. In these Examples, trichlorobenzene: C₆H₃Cl₃, which is low in toxicity and has a high boiling point, was used in place of carbon disulfide, whereby safety could be enhanced, and the pressure inside a pressure resistant vessel was lowered to 10 atm.

Specifically, first, 1 g of fullerene C₆₀ and 8.7 g of a raw material molecule ICF₂SO₂F (this mass amount is 24 times the mass amount of the fullerene, i.e., 24 equivalents per one equivalent of the fullerene) were dissolved in a mixed solvent prepared by mixing 60 ml of trichlorobenzene and 60 ml of hexafluorobenzene. After mixing, the reaction mixture was heated in an autoclave to respective temperatures of 150° C. in Example 4, 160° C. in Example 5, and 170° C. in Example 6, to effect reaction for about four days (96 hours).

After the reaction was over, the reaction mixture was cooled, and trichlorobenzene and hexafluorobenzene were distilled off from the reaction mixture at 100° C. and a reduced pressure, followed by vacuum drying at 100° C., to obtain a reaction product including the precursor molecule in the form of a dark brown powder. The reaction products were obtained in respective amounts of 2.81 g in Example 4, 3.01 g in Example 5, and 2.80 g in Example 6. The yields of the reaction were higher than that in Comparative Example 8 to be described later, probably because of the difference between the solvents used.

Upon measurement by FT-IR, not any absorption due to unreacted fullerene was observed. In addition, upon measurement by TOF-MS, also, not any peak at 720 corresponding to unreacted fullerene was observed. These results show that substantially 100% of the fullerene was reacted. Besides, it was made clear from the TOF-MS spectra that the reaction products were compounds in which a certain number of difluoro(fluorosulfonyl) groups —CF₂—SO₂F are bonded to each C₆₀ fullerene molecule, the number being 10 on average in Example 4 (reaction temperature: 150° C.), 11 on average in Example 5 (reaction temperature: 160° C.), and 10 on average in Example 6 (reaction temperature: 170° C.).

Furthermore, when the reaction was effected at 160° C. over seven days, the number of difluoro(fluorosulfonyl) groups —CF₂—SO₂F introduced to the precursor molecule was 11. This result is the same as in the case of Example 5 in which the reaction was effected at 160° C. over four days. From the comparison of these results, it is considered that, as to the reaction in the autoclave, the reaction temperature is more important than the reaction time.

Subsequently, in the same manner as in Example 1, the precursor molecule was hydrolyzed by use of a mixed solvent of an aqueous sodium hydroxide solution with THF, followed by replacement of the sodium ions with the hydrogen ions, to obtain the proton dissociating functional molecule.

(Evaluation of Proton Conductivity)

A sample of the proton dissociating functional molecule synthesized as above was vacuum dried at room temperature for 12 hours, and the resulting powder was molded by a tablet molding machine into a pellet having a thickness of about 300 μm, of which the proton conductivity in dry state was determined in the same manner as in Example 1. The sample in Example 4 had a proton conductivity in dry state of 1.8×10⁻³ Scm⁻¹, the sample in Example 5 had 2.8×10⁻³ Scm⁻¹, and the sample in Example 6 had 1.7×10⁻³ Scm⁻¹. The sample in Example 5 showed the highest proton conductivity, probably because of the largest number of the proton dissociating groups introduced per one fullerene molecule.

### Comparative Example 8

A proton dissociating functional molecule was synthesized by the existing-art method shown in Patent Document 3, at such a temperature as to enable direct comparison with Examples 1 to 6, and, upon the synthesis, the yield and the proton conductivity were determined.

First, 1 g of fullerene C₆₀ and 8.7 g of a raw material molecule ICF₂SO₂F (a mass amount of 24 times the mass amount of the fullerene, namely, 24 equivalents per 1 equivalent of the fullerene) were dissolved in a mixed solvent obtained by mixing about 79 ml of carbon disulfide and about 79 ml of hexafluorobenzene. After the mixing, the reaction mixture was heated to 160° C. in an autoclave, to effect reaction for seven days.

After the reaction was over, the reaction mixture was cooled, and carbon disulfide and hexafluorobenzene were distilled off from the reaction mixture at a reduced pressure, followed by vacuum drying at 100° C., to obtain a reaction product including the precursor molecule in the form of a dark brown powder. The reaction product was obtained in an amount of 1.6 g, the yield being 65% based on the theoretical amount.

It was made clear from the TOF-MS spectrum that the precursor molecule is a compound in which an average of eight difluoro(fluorosulfonyl) groups —CF₂—SO₂F are bonded to one C₆₀ fullerene molecule.

Subsequently, the precursor molecule was hydrolyzed by use of a mixed solvent of an aqueous sodium hydroxide solution and THF, in the same manner as in Example 1, and the sodium ions were replaced with the hydrogen ions, to obtain a proton dissociating functional molecule.

For a sample of the proton dissociating functional molecule synthesized as above, the proton conductivity in dry state was determined in the same manner as in Example 1, to be 8.9×10⁻⁴ Scm⁻¹.

A comparison of Examples 1 to 6 with Comparative Example 8 reveals that it is possible in Examples 1 to 6 to introduce larger numbers of proton dissociating groups to each fullerene molecule and, as a result, to synthesize proton dissociating functional molecules being higher in proton conductivity, as compared with the case of Comparative Example 8. This shows that according to the preparation method based on the present invention, it is possible to obtain a proton conductive material higher in quality, as compared with the case of the existing-art preparation method in which the above-mentioned second reaction is carried out at a high pressure.

The proton dissociating functional molecules synthesized in Examples 1 to 6 are materials suitable for use as a material of, for example, a proton conductor used in a fuel cell.

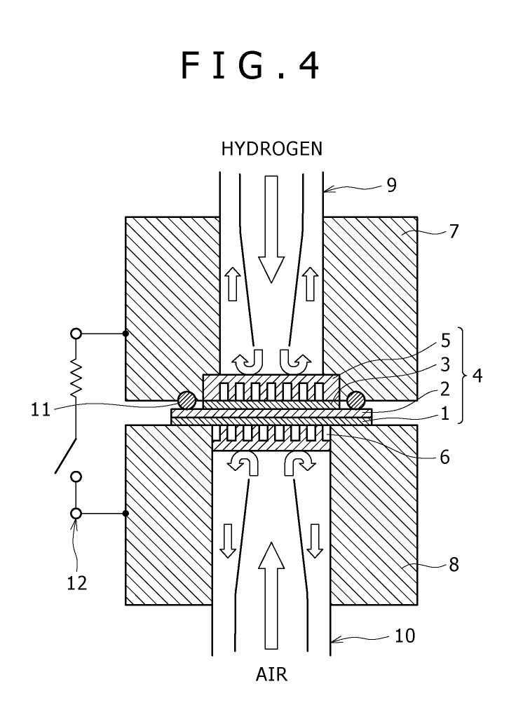

FIG. 4 is a schematic sectional view showing an example of the configuration of a fuel cell. In this electrochemical system, a proton conductor 2 formed from a proton dissociating functional molecule prepared by the preparation method based on the present invention is formed into the shape of a thin membrane, and a fuel electrode 3 and an oxygen electrode 1 are joined to the proton conductor 2 together with electrode catalysts and the like (not shown), to form a membrane-electrode assembly (MEA)₄. The membrane-electrode assembly (MEA) 4 is incorporated into the fuel cell by being clamped between a cell upper half 7 and a cell lower half 8.

The cell upper half 7 and the cell lower half 8 are provided respectively with gas supply pipes 9 and 10, and, for example, hydrogen is fed through the gas supply pipe 9, while air or oxygen is fed through the gas supply pipe 10. The gases are supplied respectively to the fuel electrode 3 and the oxygen electrode 1 through gas supply parts 5 and 6 provided with gas-passing pores (not shown). The gas supply part 5 establishes electrical connection between the fuel electrode 3 and the cell upper half 7, while the gas supply part 6 establishes electrical connection between the oxygen electrode 1 and the cell lower half 8. In addition, an O-ring 11 is disposed at the cell upper half 7 so as to prevent leakage of the hydrogen gas.

Power generation can be effected by closing an external circuit 12 connected to the cell upper half 7 and the cell lower half 8 while supplying the above-mentioned gases. In this instance, hydrogen is oxidized according to the following formula 1:

2H₂→4H⁺+4e⁻ (Formula 1)

on the surface of the fuel electrode 3, to give electrons to the fuel electrode 3. The hydrogen ions H⁺ thus produced migrate through the proton conductor 2 to the oxygen electrode 1.

The hydrogen ions having thus migrated to the oxygen electrode 1 react according to the following formula 2:

O₂+4H⁺+4e⁻→2H₂O (Formula 2)

with oxygen supplied to the oxygen electrode 1, to produce water. In this instance, oxygen takes electrons from the oxygen electrode 1, to be thereby reduced.

In this case, when the proton conductor 2 is preliminarily formed to be sufficiently thin, it is possible to moisten the proton conductor 2 with the water produced on the oxygen electrode 1, and to permit the proton conductor membrane 2 to exhibit a high proton conductivity. In addition, as compared with a proton conductor film configured by use of Nafion (tradename) according to the existing art, there are the merits that the operating temperature of the fuel cell can be raised, the fuel cell can be operated even under the conditions where moisture or water is absent, and the moisture control system for the proton conductor membrane can be unnecessitated or simplified.

In addition, the fuel electrode 3 can be supplied with methanol so as to obtain a so-called direct methanol type fuel cell.

As has been described above, according to the embodiments and examples of the present invention, a fullerene-based proton conductive material having a high proton conductivity and being thermally and chemically stable even under the conditions required of an electrochemical system can be prepared more inexpensively, more safely and in such conditions as to enable easy quality control.

While the present invention has been described above based on embodiments and examples thereof, the invention is not to be construed as limited by the embodiments and examples in any way, and appropriate modifications are naturally possible within the scope of the gist of the invention.

### INDUSTRIAL APPLICABILITY

The present invention is applicable to electrochemical devices such as fuel cells and sensors in which an ion conductor membrane is sandwiched between opposite electrodes to constitute an electrochemical reaction part. Among others, the present invention is optimally applicable to improvements in the performance and cost of fuel cells or the like through raising the operating temperature of a solid polymer electrolyte type fuel cell according to the existing art, simplifying the system for control of moisture in a membrane, or the like measure. Further, the invention is optimally applicable to configuration of a direct methanol fuel cell or the like which has been difficult to realize with a proton conductor film according to the existing art.

1: A method of preparing a raw material molecule of an ion dissociation molecule, comprising:
reacting a reactant represented by the general formula I: AgOOC—Rf—Pg, in which a precursor (-Pre) of an ion dissociating group and a silver salt of a carboxyl group are linked to each other through an at least partly fluorinated spacer group (—Rf—), with iodine to prepare a reaction product represented by the general formula II: I—Rf-Pre,
wherein in an exhaust passage for a mixed gas of said reaction product with carbon dioxide, said mixed gas is cooled to a temperature lower than the boiling point of said reaction product and higher than the freezing point of said reaction product so as to condense said reaction product into a liquid while keeping said carbon dioxide in the gaseous state, and then
said liquefied reaction product and said mixed gas are led into a trapping vessel cooled to a temperature of not higher than the boiling point of the reaction product and not lower than the subliming point of carbon dioxide, so as to trap said reaction product.

2: The method of preparing the raw material molecule of the ion dissociation molecule as set forth in claim 1, wherein silver(I) difluoro(fluorosulfonyl)acetate: AgOOCCF₂SO₂F is used as said reactant so as to prepare said reaction product including difluoroiodomethanesulfonyl fluoride: ICF₂SO₂F.

3: The method of preparing the raw material molecule of the ion dissociation molecule as set forth in claim 2, wherein said reactant is reacted with said iodine in equimolar relation.

4: The method of preparing the raw material molecule of the ion dissociation molecule as set forth in claim 3, wherein said reaction is carried out at 110° C.

5: The method of preparing the raw material molecule of the ion dissociation molecule as set forth in claim 1, wherein said exhaust passage is cooled to −15° C., and said trapping vessel is cooled with dry ice.

6: A method of preparing an ion dissociation molecule, comprising:
synthesizing a reaction product of said general formula II by a preparation method as set forth in any of claims 1 to 5; and
synthesizing a precursor molecule represented by the general formula III: Cm(—Rf-Pre)n, where m is a natural number such that Cm can form a fullerene, and n is a natural number, by a second reaction between a fullerene molecule and said reaction product in a solvent having at least one of a boiling point of not lower than 150° C. and at a normal pressure or a reduced pressure, and hydrolyzing said precursor group (-Pre) of said precursor molecule so as to convert said precursor group into an ion dissociating group.

7: The method of preparing the ion dissociation molecule as set forth in claim 6, wherein the reaction temperature of said second reaction is 150° C. to 300° C., and the reaction solvent for said second reaction is a halobenzene.

8: The method of preparing the ion dissociation molecule as set forth in claim 6 or 7, wherein the reaction temperature of said second reaction is 150° C. to 300° C., and the reaction solvent for said second reaction includes at least one solvent selected from the group consisting of trichlorobenzene, n-propylbenzene, isopropylbenzene, n-butylbenzene, sec-butylbenzene, tert-butylbenzene, o-dibromobenzene, m-dibromobenzene, o-dichlorobenzene, m-dichlorobenzene, 1-phenylnaphthalene, and 1-chloronaphthalene.

9: The method of preparing the ion dissociation molecule as set forth in claim 7, wherein said solvent includes 1,2,4-trichlorobenzene used as a single solvent.

10: The method of preparing the ion dissociation molecule as set forth in claim 7, wherein a mixed solvent obtained by mixing trichlorobenzene and hexafluorobenzene in a volume ratio of 1:1 is used as a reaction solvent for said second reaction.

11: The method of preparing the ion dissociation molecule as set forth in claim 6, wherein said reaction product is slowly added dropwise to a solution obtained by dissolving said fullerene molecule in said solvent, according to the progress of said second reaction.

12: The method of preparing the ion dissociation molecule as set forth in claim 11, wherein said second reaction is carried out by continuing stirring even after said dropwise addition.

13: The method of preparing the ion dissociation molecule as set forth in claim 6, wherein said fullerene molecule is Cm, where m=36, 60, 70, 76, 78, 80, 82, 84, 90, 96, or 266.

14: The method of preparing the ion dissociation molecule as set forth in claim 13, wherein said fullerene molecule is C₆₀ or C₇₀.

15: The method of preparing the ion dissociation molecule as set forth in claim 6, wherein a glass-made vessel is used as a reaction vessel for said second reaction.

16: The method of preparing the ion dissociation molecule as set forth in claim 6, wherein a vessel having a metallic surface lined with a glass layer is used as a reaction vessel for said second reaction.

17: The method of preparing the ion dissociation molecule as set forth in claim 6, further comprising the step of replacing an ion bonded to said ion dissociating group produced upon said hydrolyzing step with a predetermined ion so as to obtain a predetermined ion dissociation molecule.

18: The method of preparing the ion dissociation molecule as set forth in claim 17, wherein a proton dissociating molecule is obtained as said ion dissociation molecule.

19: The method of preparing the ion dissociation molecule as set forth in claim 17, wherein said ion dissociating group is a proton dissociating group selected from the group consisting of the hydrogen sulfate ester group —OSO₂OH, the sulfonic group —SO₂OH, the dihydrogenphosphoric ester group —OPO(OH)₂, the monohydrogenphosphoric ester group —OPO(OH)—, the phosphono group —PO(OH)₂, the carboxyl group —COOH, the sulfonamide group —SO₂—NH₂, the sulfonimide group —SO₂—NH—SO₂—, the methanedisulfonyl group —SO₂—CH₂—SO₂—, the carboxamide group —CO—NH₂, and the carboximide group —CO—NH—CO—.
